(19) United States
(12) Patent Application Publication
(10) Pub. No.: US 2010/0049329 A1
(43) Pub. Date: Feb. 25, 2010

(54) Coxo-femoral surface prosthesis for veterinary use

(76) Inventor: Alessandro Vio, Tortona(AL) (IT)

Correspondence Address: JACOBSON HOLMAN PLLC, 400 SEVENTH STREET N.W., SUITE 600, WASHINGTON, DC, 20004, US

(21) Appl. No.: 12/458,811

(22) Filed: Jul. 23, 2009

(30) Foreign Application Priority Data
Jul. 25, 2008 (IT) MI2008A 001367
Dec. 12, 2008 (IT) MI2008A 002207

(51) Int. Cl. A61F 2/36

(52) U.S. Cl. 623/23.12

(57) ABSTRACT

A coxo-femoral surface prosthesis for veterinary use is described. The prosthesis comprises a femoral cap and an acetabular cup which are complementary in volume, an intermediate lining and a screw fixing femoral stem. The femoral stem comprises a head portion, a shank portion and a threaded portion. (FIG. 1)

The present invention concerns a coxo-femoral surface prosthesis for veterinary use, in particular for dogs.

The application of coxo-femoral prostheses, also known as hip prostheses, to dogs is required for reconstructive surgical operations on dogs which have undergone traumas to the coxo-femoral joint, and also on dogs whose joint has suffered irreversible degeneration of other types, such as those consequential on hip dysplasia.

In the current state of the art, veterinary methods of reconstructing the coxo-femoral joint provide for the use of a stem prosthesis consisting of a femoral part and an acetabular part. The femoral part of the prosthesis replaces the head and neck of the femur, which must undergo osteotomy (resection) to enable it to be applied. The femoral part of the prosthesis furthermore terminates in a femoral stem which serves to anchor the femoral part of the prosthesis to the dog's femur; following drilling and boring of the femoral canal, the femoral stem is inserted into the patient's femoral canal and fixed by the use of orthopaedic pins or surgical cement.

The operation is of a certain gravity and inevitable bloodiness, and requires high professionalism and suitable equipment to carry it out, and a long and demanding post-operative period for the dog and its owner: the dog, in fact, should not stress the limb for several weeks, in order to favour substantial bone regrowth, before putting weight on the limb again. This is difficult to achieve even from the best-trained animal, and constant surveillance is therefore required. Given the major character of the operation, considerable use is made of post-operative drugs, such as painkillers and antibiotics.

In the field of human prosthetic reconstruction of the coxo-femoral joint, surface prostheses are currently applied, consisting of the combination of a femoral cap and an acetabular cup. Surface prostheses allow the neck and the head of the femur to be retained, since the cartilaginous cover of the head of the femur is removed and the femoral cap is applied, re-covering the head of the femur; fixing is by means of pins, cemented or not cemented, inserted under pressure in a canal excavated in the femur with extreme precision, by means of computerized equipment.

An acetabular cup is inserted under pressure (“press fit” technique) into the acetabulum, possibly fixed by anchoring screws.

The femoral cap thus slides on the acetabular cup to reconstruct the joint.

Surface prostheses are not new in themselves: after the first experiments in the 1970s they were abandoned because of problems of premature wear of the materials that were used at the time; they were then successfully rediscovered in more recent times, thanks to progress in the technology.

As may be guessed, an operation to implant a surface prosthesis is much less bloody than the implant of a conventional prosthesis, and requires a much shorter and more easily tolerated post-operative course; for this reason the use of this method, where possible, and the corresponding equipment is widespread in the human field.

In the veterinary field it is not possible to apply the same identical technology as the surface prostheses known in the current state of the art, firstly because in veterinary medicine the techniques and the corresponding high-precision machinery are not available to enable the same method to be followed; in addition, cement cannot be used for fixing the femoral cap to the head of the dog's femur, because the levels of bacterial charge are higher in the veterinary field, and the possibility of infection in the cemented area, and therefore of rejection of the prosthesis, is much higher.

The object of the present invention is therefore to create a veterinary coxo-femoral surface prosthesis with good implantability characteristics (low probability of rejection) and with implant surgery that is much less bloody than is required for a conventional prosthesis.

In accordance with the invention, this object is achieved with a coxo-femoral surface prosthesis for veterinary use, characterized by comprising a femoral cap and an acetabular cup which are complementary in volume, and a screw fixing femoral stem.

In one embodiment of the invention the aforesaid femoral prosthesis is furthermore characterized in that the contact surface of the acetabular cup with the femoral cap defines spaces which are complementary in volume to each other.

These and other characteristics of the present invention will be made more clearly evident from the following detailed description of examples of its practical embodiment which are illustrated without limiting effect in the attached drawings, in which:

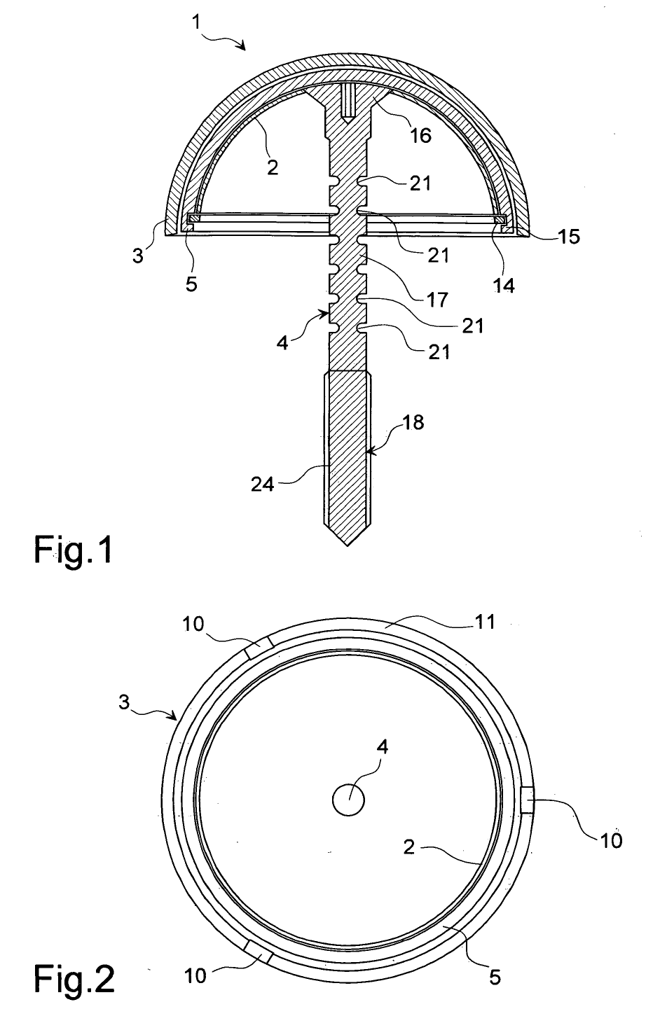

FIG. 1 shows a view in axial section of an assembled coxo-femoral prosthesis;

FIG. 2 shows a view from below of a femoral cap, an intermediate lining and an acetabular cup fitted together;

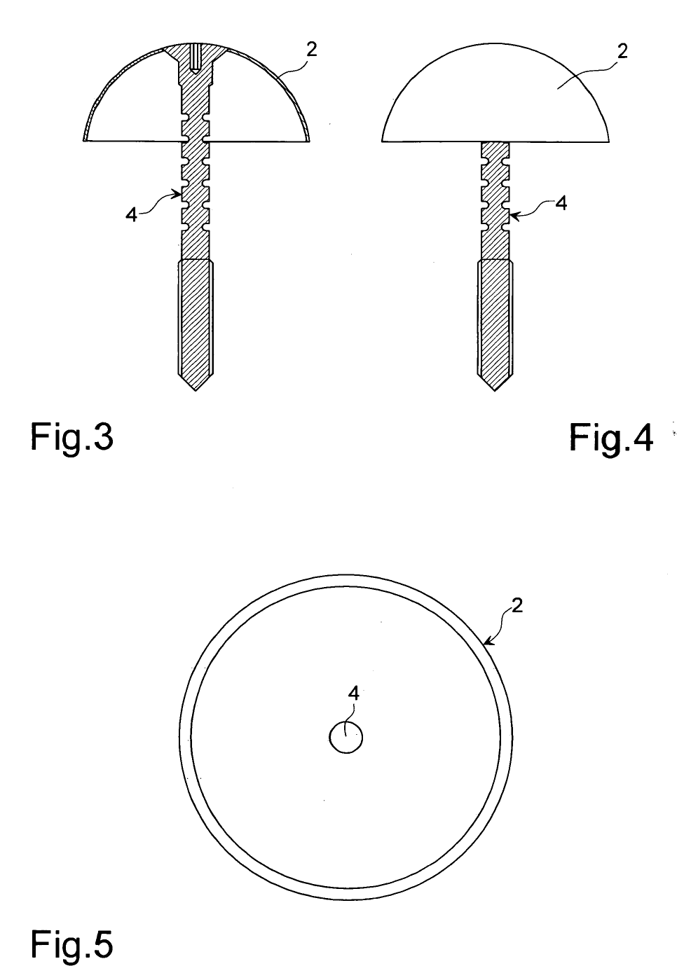

FIG. 3 shows a view in axial section of a femoral stem and a femoral cap assembled together;

FIG. 4 shows a lateral view, partially sectioned, of FIG. 3;

FIG. 5 shows a view from below of the femoral cap;

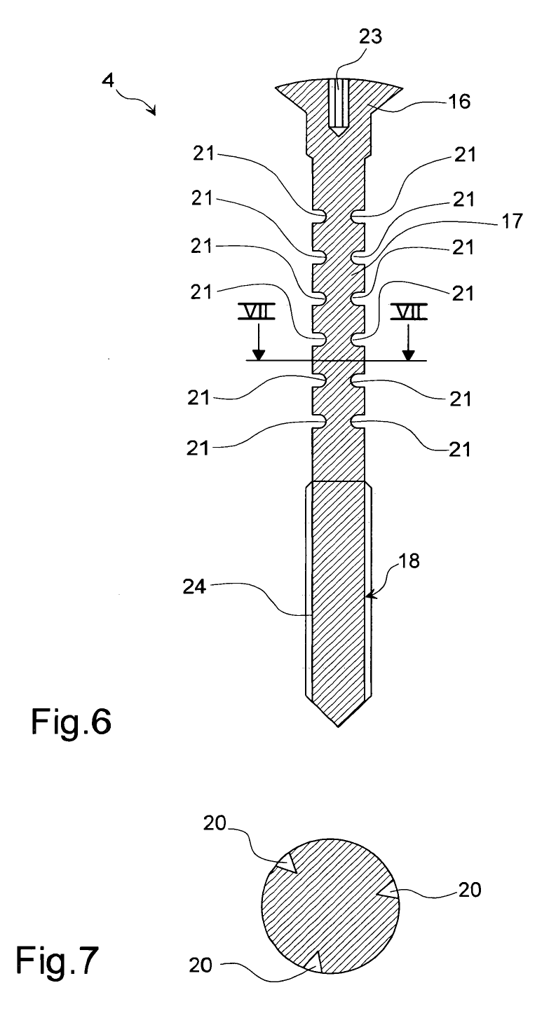

FIG. 6 shows an axial section of the femoral stem;

FIG. 7 shows a transverse section of the femoral stem along the line VII-VII in FIG. 6;

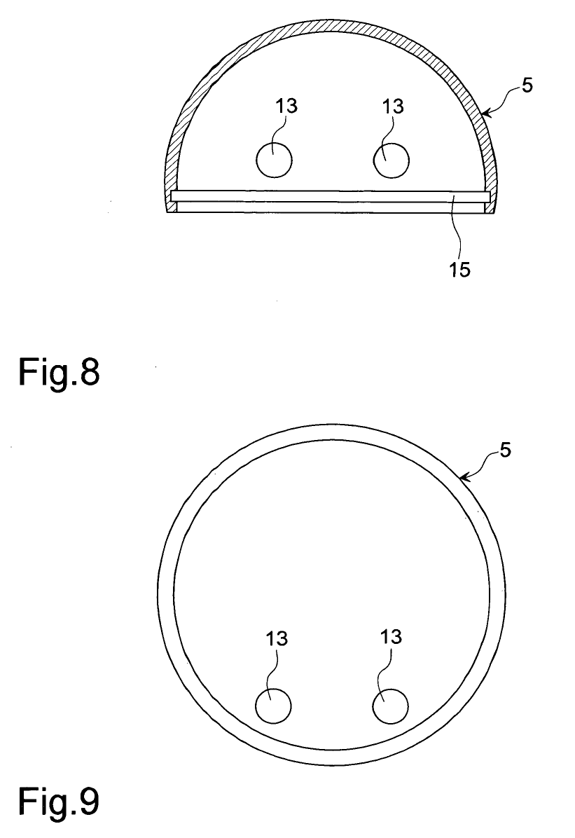

FIG. 8 shows an axial section of the intermediate lining;

FIG. 9 shows a view from below of the intermediate lining;

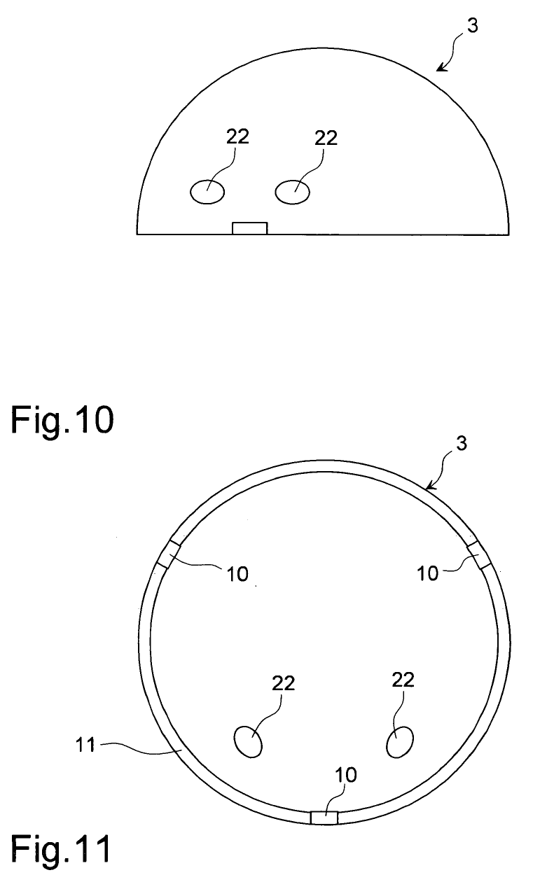

FIG. 10 shows a lateral view of the acetabular cup;

FIG. 11 shows a view from below of the acetabular cup;

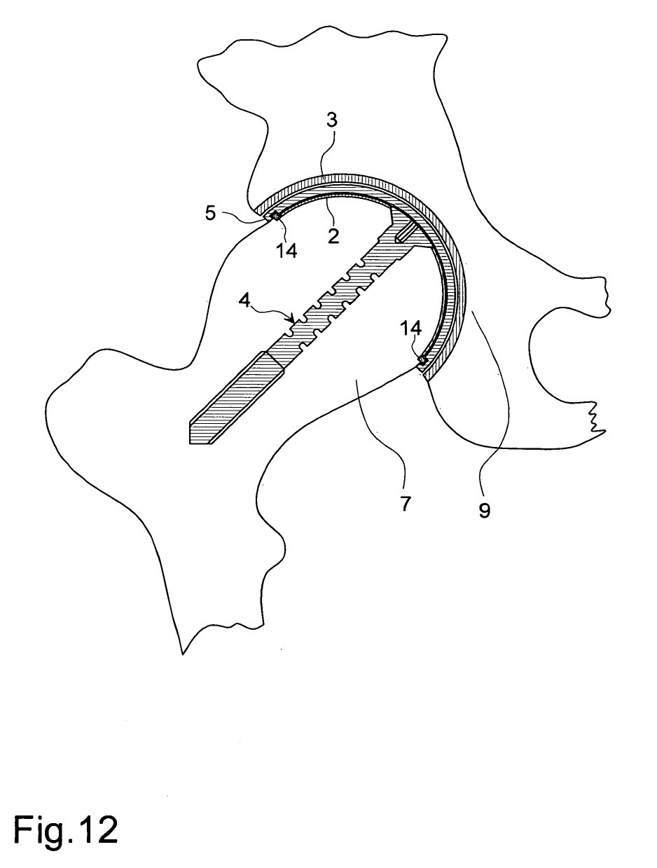

FIG. 12 shows a view in section of the implanted coxo-femoral prosthesis;

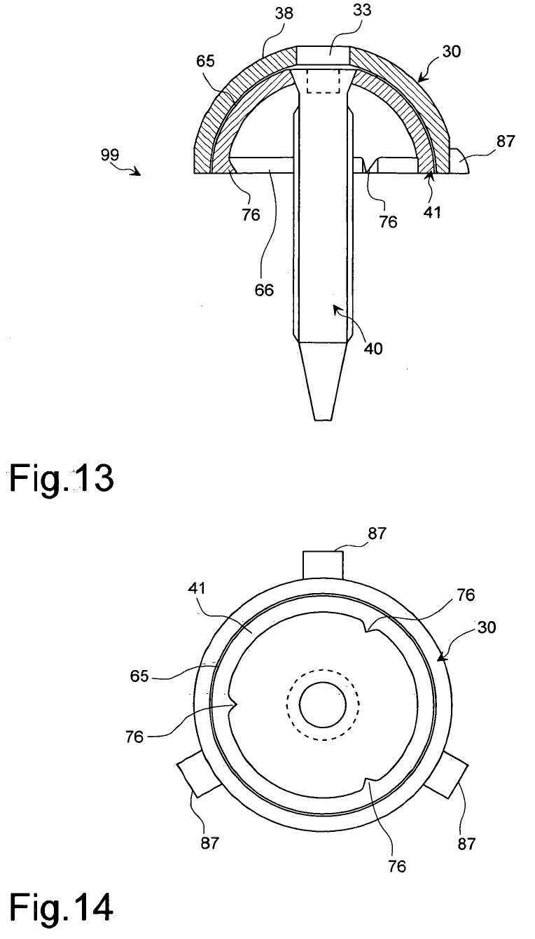

FIG. 13 shows a view in axial section of an assembled coxo-femoral prosthesis comprising a femoral cap and an acetabular cup of a second embodiment, and a femoral screw;

FIG. 14 shows a view from below of the femoral cap and the acetabular cup of the second embodiment fitted together;

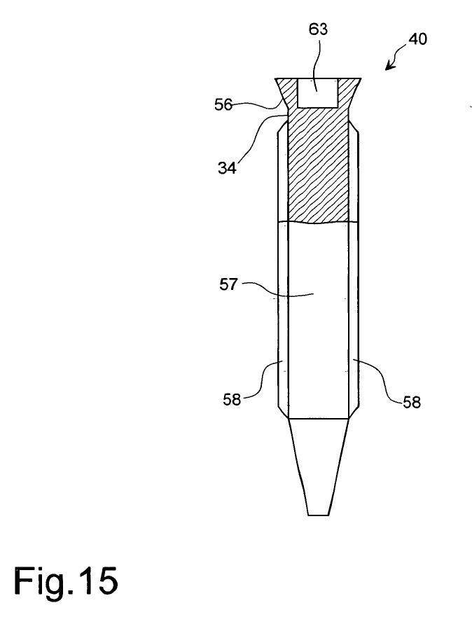

FIG. 15 shows a lateral view, with the head in partial section, of a second embodiment of a femoral screw;

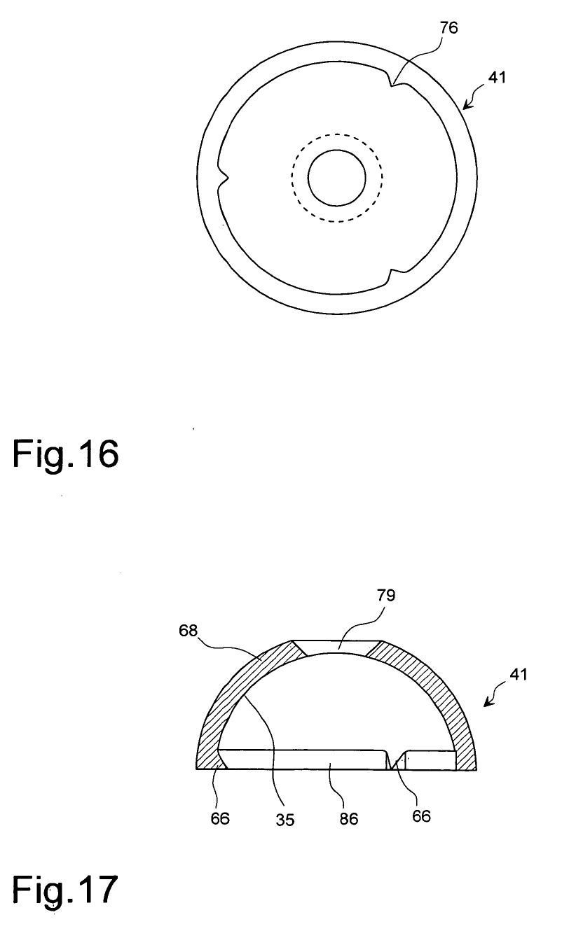

FIG. 16 shows a view from below of the second embodiment of the femoral cap;

FIG. 17 shows a view in vertical section of the second embodiment of the femoral cap;

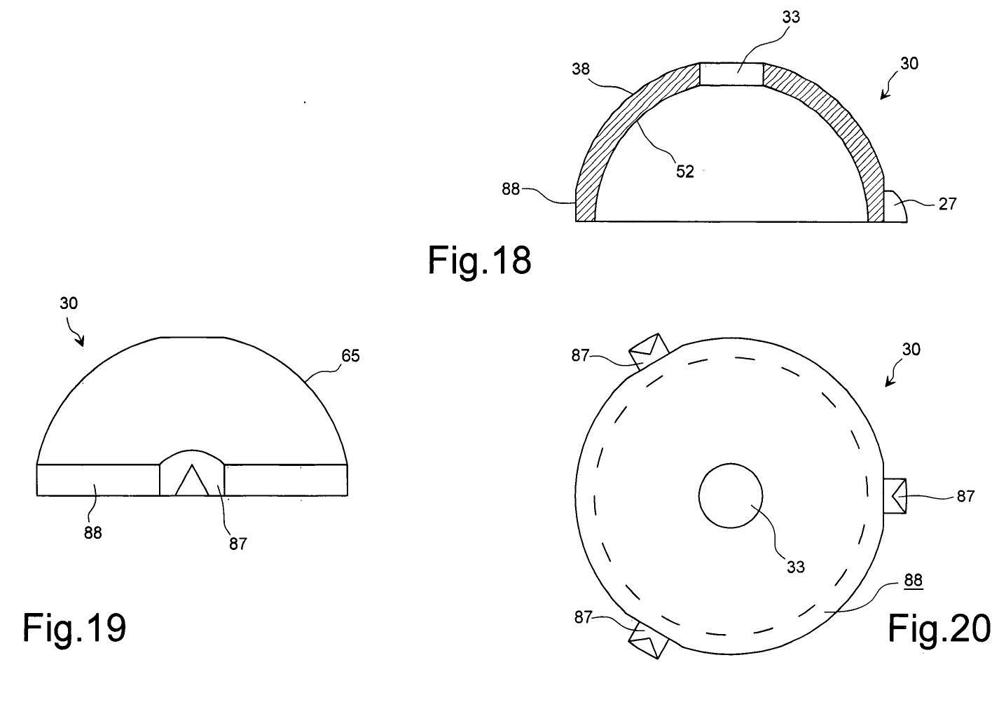

FIG. 18 shows a view in vertical section of a second embodiment of the acetabular cup;

FIG. 19 shows a lateral view of the second embodiment of the acetabular cup;

FIG. 20 shows a view from above of the second embodiment of the acetabular cup;

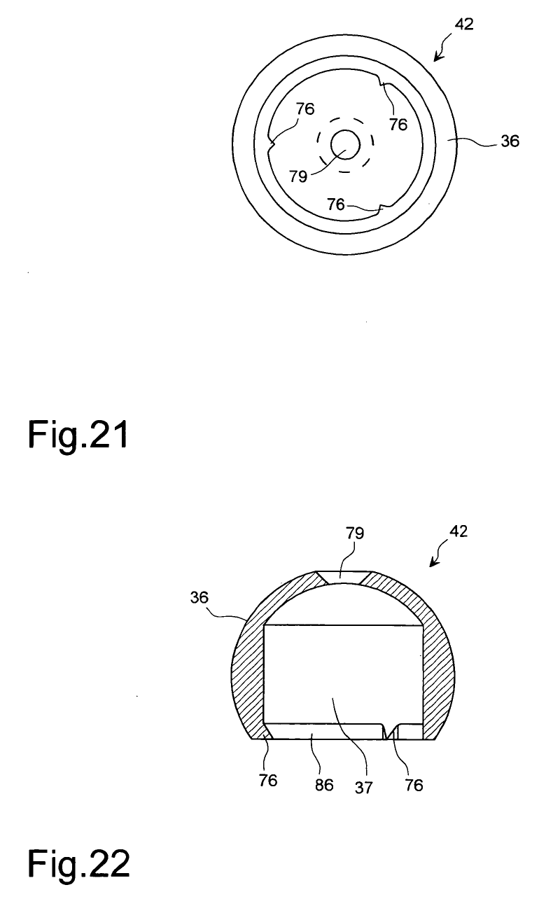

FIG. 21 shows a view from below of a third embodiment of the femoral cap;

FIG. 22 shows a view in vertical section of the third embodiment of the femoral cap;

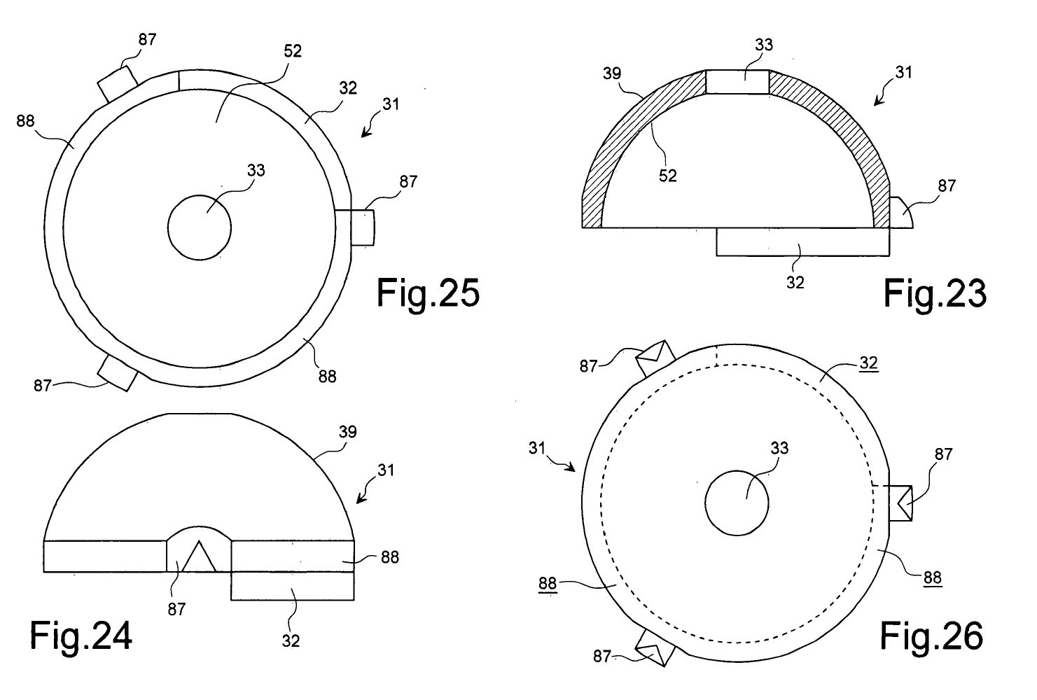

FIG. 23 shows a view in vertical section of a third embodiment of the acetabular cup;

FIG. 24 shows a lateral view of the third embodiment of the acetabular cup;

FIG. 25 shows a view from above of the third embodiment of the acetabular cup;

FIG. 26 shows a view from below of the third embodiment of the acetabular cup;

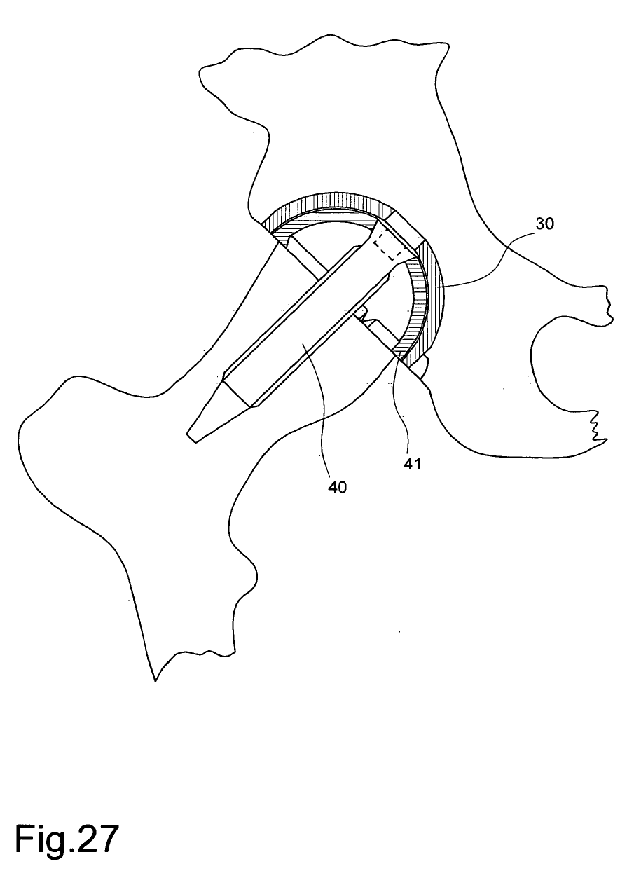

FIG. 27 shows a view in section of the second embodiment of the implanted coxo-femoral prosthesis.

With reference to FIGS. 1-12 and in particular to FIG. 1, it should be noted an assembled coxo-femoral prosthesis 1, comprising a femoral cap 2 with a relative screw fixing femoral stem 4, an acetabular cup 3 and an intermediate lining 5 which is provided with an annular housing 15 for a locking ring 14 (FIG. 8), such as a Seeger ring, for assembling the femoral cap 2 to the intermediate lining 5. Other alternative locking means, not shown, can be used.

In FIG. 12 the assembled components of the prosthesis I can be seen: the femoral stem 4 locks onto a femoral head 7 the femoral cap 2, which is held in the intermediate lining 5 by the locking ring 14. The intermediate lining 5 rotates freely, about three axes perpendicular to each other, in the acetabular cup 3, which is fixed by two screws, not shown, to an acetabulum 9.

The acetabular cup 3 (FIGS. 10 and 11), which is housed in the acetabulum 9, has the shape, mirror-finished to reduce friction particularly on the inner surface, of a spherical portion slightly exceeding the hemisphere, and is provided with fixing holes 22 for fixing means to the acetabulum 9. The screws do not protrude from the inner surface of the acetabular cup, so as not to obstruct the rotation between cup 3 and intermediate lining 5.

The acetabular cup 3 is provided furthermore with positioning means 10. In the present embodiment, the positioning means are three notches 10 (FIGS. 10 and 11), spaced 120° apart from each other, located on an edge 11 of the acetabular cup 3. The notches 10 serve, in the course of the surgical operation, for positioning the acetabular cup 3 in the acetabulum 9, by means of a positioner, not shown.

The surface of the acetabular cup 3 which is in contact with the acetabulum 9 is kept rough from the casting so as to favour biological anchorage by means of osteosynthesis, as a result of which the bone grows into the irregularities of the surface.

Before the operation, the acetabular cup 3 and the intermediate lining 5 are provided already fitted to each other by hot assembly. As will become clearer in what follows, their shape, a portion of a sphere which exceeds the maximum diameter, would not allow assembly during the course of the surgical operation.

The intermediate lining 5 (FIGS. 8 and 9) is shaped internally as a hemisphere as far as the maximum diameter, and then the internal profile continues with a cylindrical profile, matched to the maximum diameter (FIG. 8), to permit the insertion of the femoral cap 2, during the operation. The hemispherical internal surface of the intermediate lining 5 is complementary in volume to the external surface of the femoral cap 2. Below the maximum diameter of the hemispherical part of the intermediate lining 5, an annular housing 15, i.e. a circumferential groove, is capable, as a result of its dimensions and position, of accommodating the locking ring 14, for fixing the femoral cap 2 inside the intermediate lining 5. The femoral cap 2 and the intermediate lining 5, after fixing with the Seeger ring, are integral with each other, for which reason only modest rotations are possible on the locking ring 14, and these do not prejudice the operation of the joint.

The external surface of the intermediate lining 5 is complementary with respect to the contact surface with the acetabular cup 3, and has a mirror finish to reduce friction.

The intermediate lining 5 is furthermore provided with two holes 13, for the passage of the fixing means 8, typically screws, to fix the acetabular cup 3 to the acetabulum 9. The screws do not interfere with the intermediate lining 5, which is rotatable about three axes perpendicular to each other, with respect to the acetabular cup 3. The holes 13 serve only for the passage of the screws. As was indicated earlier and as will become clearer in what follows, the intermediate lining 5 and the acetabular cup 3 are hot-assembled before the operation, and at the end of the operation the intermediate lining 5 and the acetabular cup 3 are contained one within the other and rotate without any play between them about three axes perpendicular to each other, to recreate the movements of the coxo-femoral joint.

The femoral stem 4 is provided with a head portion 16, a shank portion 17 and a threaded portion 18.

The head portion 16 is provided with a hexagonal recess 23 (FIG. 6) suitable for allowing the stem 4 to be tightened in the head of the femur 7 by means of a suitable alien key. The shank portion 17 is provided with grooves 19 (shown only in embodiments 20 and 21), in particular longitudinal grooves 20, with a triangular profile in transverse section to the groove (FIG. 7), and annular grooves 21, with a semicircular profile in transverse section to the groove. Said grooves 20, 21 have the function of favouring biological anchorage by osteosynthesis: the bony tissue of the femur grows in time into the grooves 20, 21, improving the resistance to axial and torsional loads on the femoral stem 4. Advantageously, the grooves can be three longitudinal grooves 20, and six annular grooves 21.

The threaded portion 18 carries a thread 24 which promotes an action of mechanical gripping right from the time of the implant.

The femoral cap 2 (FIGS. 2-4) has a hemispherical shape externally (and typically also internally), and is provided with a hole 6 for the passage of the femoral stem 4 for fixing to the head of the femur 7 by means of the femoral stem 4. The surface in contact with the head of the femur 7 is rough from the casting, so as to favour a certain degree of osseous anchorage through osteosynthesis.

The material for all the components can be titanium, or stainless steel 316, which both guarantee biocompatibility.

In the course of the operation, after removing the cartilage from the head of the femur 7, the femoral cap 2 is fixed to the head of the femur 7 by means of the femoral stem 4. After milling of the acetabulum to adapt it to the external dimensions of the acetabular cup 3, the set consisting of acetabular cup 3 and intermediate lining 5, which have previously been hot-assembled, is inserted into the acetabulum, positioning them by means of the notches 10. By rotating the intermediate lining 5, the through holes 13 located in the lining 5 are aligned with the fixing holes 22, situated on the acetabular cup 3, to allow the screws to pass through, and these are used to fix the acetabular cup 3 to the acetabulum 9. The screws disappear into the acetabular cup 3 while locking it into place, without causing interference with the intermediate lining 5. The femur with the femoral cap 2 fitted to the head of the femur 7 is then brought close to the intermediate lining 5, and the femoral cap 2 is placed inside the intermediate lining 5. At this point the locking ring 14 is released into the groove, and the femoral cap is constrained within the intermediate lining 5.

The mechanical part of the coxo-femoral joint has thus been reconstructed.

The technique described enables a brief post-operative course, with the possibility of controlled movement, with the dog on a lead, as early as two weeks after the operation, compared with about two months after an operation with the standard prosthesis.

To complete the description, some typical measurements of the various components of the prosthesis 1 are added here.

The acetabular cup 3 has a maximum diameter of 39 mm, a height of 22 mm and fixing holes 22 located at 7 mm from the edge 11 and spaced at 15 mm apart from each other.

The orthopaedic cortical screws are typically 3.5 mm long.

The notches 10 are 2 mm deep and 5 mm long.

The intermediate lining 5 has an external diameter of 32.9 mm and a thickness of 1.95 mm, with through holes 13 in similar positions to the acetabular cup 3, with a maximum diameter of 5 mm. The annular housing 15 is located at 1.5 mm from the edge, and is 1 mm wide and 1 mm deep.

The femoral cap 2 has an external diameter of 29 mm and a thickness of 2 mm.

The femoral stem 4 is 45 mm long.

The dimensions of the cup 3, lining 5 and femoral cap 2 vary with the weight of the dog, according to the table for matching components below. The stem has the same dimensions irrespective of the size of the dog.

| | | | |
|---|---|---|---|
| External diameter | 39 | 37 | 35 |
| acetabular cup 3 (mm) | | | |
| External diameter | 32.9 | 30.9 | 28.9 |
| Intermediate lining 5 (mm) | | | |
| External diameter | 29 | 27 | 25 |
| Femoral cap 2 (mm) | | | |

With reference to FIGS. 13-27 and in particular to FIGS. 13 and 14, it should be noted a further embodiment of the assembled coxo-femoral prosthesis 99, comprising a second embodiment of femoral cap 41 with a relative screw fixing femoral stem 40, and a second embodiment of acetabular cup 30. The inner surface 52 of the acetabular cup 30 is hemispherical like the outer surface 68 of the femoral cap 41, which is complementary to it, as may be appreciated from the view from below shown in FIG. 14. The contact surface 65 of the acetabular cup 41 with the femoral cap 30 defines spaces which are complementary in volume to each other.

In FIG. 15 it should be noted the femoral stem 40 provided with a collar 34 and a shank portion 57 with a thread 58 which exceeds in diameter the collar 34. A head 56 of the stem 40 is provided with a recess 63 for tightening the stem 40 with a square key or allen key. The shank, which is entirely threaded, is made up of an intermediate cylindrical part and a tapered end part, in order to allow easier insertion into the femur.

In FIGS. 16 and 17 it should be noted the femoral cap 41 in the second embodiment which comprises the hemispherical outer surface 68, a hemispherical inner surface 35 (FIG. 17) provided with three fastening teeth 66 arranged on an inner edge 86 of the femoral cap 30, spaced at 120° from each other (other arrangements and numbers of teeth 26 are possible) and a femoral hole 79 for the passage of the femoral stem 40 for fixing to the head of the femur.

In FIGS. 18, 19, 20 it should be noted the acetabular cup 30 in the second embodiment which comprises a hemispherical inner surface 52, complementary to the outer surface 68 of the femoral cap 41 with which it makes contact on the contact surface 65, which for this embodiment coincides with surfaces 68 and 52. A threaded hole 33 is formed in the cup 30 for fixing the acetabular cup 30 to the socket. Further acetabular holes 33 can be formed in various positions for fixing the cup 30, but are not illustrated here. Three fastening appendages 27 are arranged along an outer edge 8 of the acetabular cup 30, spaced 120° apart from each other. The number and arrangement of the fastening appendages 27 can vary.

For assembling the second embodiment, the acetabular cup 30 is positioned in the acetabulum which has been cleared of cartilage; the fastening appendages 27 have the double function of positioning the acetabular cup 30 accurately in three dimensions in the acetabulum, and of constraining the cup 30 in rotation to the acetabulum itself, to give the prosthesis a better overall grip and stability. The acetabular cup 30 is inserted under pressure (“press fit” technique), and can possibly be fixed by any known osteosynthesis screws, to be inserted in one or more acetabular holes 33 of the cup 30.

A pilot hole (not shown) is made in the head of the femur (which has been cleared of cartilage), of a diameter slightly smaller than the femoral stem 40, and of a length slightly greater than the femoral stem 40, and the femoral cap 41 is positioned and fixed with the femoral stem 40, which is screwed into the bone, guided by the pilot hole. The fastening teeth 76 prevent the rotation of the femoral cap 41 in its housing on the head of the femur, to the advantage of the stability of the implant.

In FIG. 27 the components of the prosthesis 99, according to the second embodiment, can be seen implanted: the femoral stem 40 locks the femoral cap 41 to the head of the femur. The acetabular cup 30 and the femoral cap 41 rotate freely against each other, about three axes perpendicular to each other.

FIG. 27 does not show the joint capsule and the bundles of muscles which protect the joint thus reconstructed from luxation.

In FIGS. 21 and 22 it should be noted a third embodiment for femoral cap 42, wherein the outer surface 36 of the femoral cap 42 is a portion of spherical lining which is such as to exceed the maximum diameter. The hollow internal volume, defined by an inner surface 37, is definable as a cylindrical portion surmounted by a spherical portion of lining. The femoral cap 42 also is provided with three fastening teeth 26 arranged on an inner edge 86 of the femoral cap 42 at 120° apart from each other (other arrangements and numbers of teeth 76 are possible), and with a femoral hole 79 for the passage of the femoral stem 40. Assembly of the femoral cap 42 of the third embodiment is performed after moderate milling of the head of the femur into the shape of the hollow internal volume of the femoral cap 42, which may be done with a suitable cup wheel cutter, of a shape complementary to the hollow internal volume of the femoral cap, and leaving a portion of spherical lining, which is the natural shape of the head of the femur.

In FIGS. 23, 24, 25 and 26 it should be noted a third embodiment for the acetabular cup 3 1, which has an extended portion 32 of outer edge 88, to avoid inadequate acetabular containment. Advantageously, this cup, too, is provided with an acetabular hole 33 for fixing the acetabular cup 31 to the socket. Further acetabular holes 33, including holes in various positions, can be formed for fixing the cup 31, but are not illustrated here. Three fastening appendages 87 are arranged along an outer edge 88 of this embodiment of acetabular cup 31 too, spaced 120° apart from each other. The number and arrangement of the fastening appendages 87 may vary.

It must be emphasized here that every combination is possible between different embodiments of acetabular cups 30 and 31 and femoral caps 41 and 42. For example, it is possible to assemble an acetabular cup 30 from the second embodiment with a femoral cap 42 from the third embodiment.

Assembly of the third embodiment of the acetabular cup 31 is performed in similar manner to what we have seen for the acetabular cup 30 of the second embodiment, but positioning the extended portion 32 of the outer edge 88 towards the dorsal edge of the joint, anatomically defined as the dorsal acetabular rim.

It must also be emphasized that the femoral cap 42 of the third embodiment, in combination with the acetabular cup 30 or 31 of the second or third embodiment, allows a greater excursion of the coxo-femoral joint, compared with the femoral cap 41 of the second embodiment.

Advantageously, the stem 40 and the internal surface 35, 37 of the femoral cap 41, 42 are coated with calcium hydroxyapatite, to stimulate bone growth.

An outer surface 38, 39 (FIGS. 13, 18, 19, 23, 24) of the acetabular cup 30, 31 is covered with a layer of calcium hydroxyapatite and titanium dioxide; the latter, being porous, favours osseous anchorage. The coating treatments described may be susceptible to modifications connected with technological innovation in the field.

1. Coxo-femoral surface prosthesis for veterinary use, comprising a femoral cap and an acetabular cup and an intermediate lining to facilitate assembly of the femoral cap to the acetabular cup, the femoral cap, the acetabular cup and the lining, cap having contact surfaces which are complementary in volume, and a screw fixing femoral stem, wherein the intermediate lining has, externally, the shape of a hemispherical portion exceeding the hemisphere and substantially complementary in volume to the acetabular cup and, internally, has a hemispherical shape and, for the portion exceeding the hemisphere, has a matched cylindrical profile, to facilitate the insertion of the femoral cap, which is substantially complementary in volume to it for the hemispherical part, and said intermediate lining being provided with means for locking the femoral cap into the intermediate lining, and with through holes for the passage of means for fixing the acetabular cup to the acetabulum.

2. Coxo-femoral prosthesis according to claim 1, wherein the femoral cap has an external hemispherical shape, and is provided with at least one hole for fixing to the head of the femur by means of the femoral stem.

3. Coxo-femoral prosthesis according to claim 1, wherein the acetabular cup has, internally, the shape of a hemispherical portion exceeding the hemisphere and is provided with fixing holes for means for fixing to the acetabulum, and with positioning means.

4. Coxo-femoral prosthesis according to claim 3, wherein the positioning means are notches or protrusions on an edge of the acetabular cup.

5. Coxo-femoral prosthesis according to claim 1, wherein said locking means for the intermediate lining comprise a locking ring and an annular housing with position and size suitable for accommodating said locking ring for fixing the femoral cap into the intermediate lining.

6. Coxo-femoral prosthesis according to claim 1, wherein said fixing femoral stem comprises a head portion, a shank portion and a threaded portion, and on the shank portion there are grooves for favouring biological anchorage by means of osteosynthesis.

7. Coxo-femoral prosthesis according to claim 6, wherein said grooves in said fixing stem comprise longitudinal grooves.

8. Coxo-femoral prosthesis according to claim 6, wherein said grooves in said fixing stem comprise annular grooves.

9. Coxo-femoral surface prosthesis for veterinary use, comprising a femoral cap with a top provided with a hole for passage of a fixing screw and a base opening provided with teeth for hooking to a femur head, and an acetabular cup with an internal surface complementary in volume to a corresponding external surface part of the femoral cap, and a fixing screw insertable into said hole of the top of the femoral cap for fixing the femoral cap to a femur head.

10. Prothesis according to claim 9, wherein the external surface of the femoral cap is hemispherical, with a portion extending beyond the section of maximum diameter.

11. Prosthesis according to claim 11, characterized in that the femoral cap has a plurality of fastening teeth along an inner edge.

12. Prosthesis according to claim 9, wherein the fixing screw and the internal surface of the femoral cap are coated with calcium hydroxyapatite.

13. Prosthesis according to claim 9, wherein the external surface of the acetabular cup is coated with calcium hydroxyapatite and titanium dioxide.
